(19) United States
(12) Patent Application Publication
(10) Pub. No.: US 2025/0125036 A1
(43) Pub. Date: Apr. 17, 2025

(54) MEAL RECOMMENDATION APPARATUS, MEAL RECOMMENDATION METHOD, AND RECORDING MEDIUM

(71) Applicant: NEC Corporation, Minato-ku, Tokyo (JP)

(72) Inventors: Yoji MORI, Tokyo (JP); Ayako Hoshino, Tokyo (JP); Yuya Endo, Tokyo (JP); Yuuki Watanabe, Tokyo (JP); Naruto Yajima, Tokyo (JP)

(73) Assignee: NEC Corporation, Minato-ku, Tokyo (JP)

(21) Appl. No.: 18/682,508

(22) PCT Filed: Sep. 15, 2021
(86) PCT No.: PCT/JP2021/033836

§ 371 Date: Feb. 9, 2024

(51) Int. Cl. G16H 20/60

(52) U.S. Cl. CPC G16H 20/60

(57) ABSTRACT

In order to recommend a meal menu suitable for a user, a meal recommendation apparatus (1) includes: a reception section (11) for receiving physical information or a health condition of a subject user and a request pertaining to a meal menu; a generation section (12) for generating response information including information pertaining to a meal menu which corresponds to the physical information or health condition of the subject user based on the request and the health condition of the subject user using a learned model which has learned pieces of physical information or health conditions of a plurality of second users and meal order histories of the plurality of second users; and an output section (13) for outputting the response information.

### TECHNICAL FIELD

The present invention relates to a meal recommendation apparatus and the like for recommending a meal menu to a user.

### BACKGROUND ART

Conventionally, development of a technique for recommending, by a computer, a product or a service to a user has been in progress. For example, Patent Literature 1 below discloses a recipe management server which identifies a user and recommends a category of food based on meal information indicating meals which the user and a family of the user have had in the past.

### CITATION LIST

### Patent Literature

[Patent Literature 1]
Japanese Patent Application Publication Tokukai No. 2020-64666

### SUMMARY OF INVENTION

### Technical Problem

According to the technique of Patent Literature 1, a category of food which a user and a family thereof have more frequently had is recommended. However, the user recognizes such a category of food which the user and the family thereof have more frequently had, without the need for using the technique of Patent Literature 1. That is, in the technique of Patent Literature 1, there is room for improvement in that it is difficult to recommend a category that the user himself/herself does not recognize but that conforms to preference of the user.

An example aspect of the present invention is accomplished in view of the above problem, and an example object thereof is to provide a technique that makes it possible to recommend a meal menu which is suitable for a user even if the user himself/herself does not recognize suitability.

### Solution to Problem

A meal recommendation apparatus in accordance with an example aspect of the present invention includes: a reception means for receiving physical information or a health condition of a subject user and a request pertaining to a meal menu; a generation means for generating response information including information pertaining to a meal menu which corresponds to the physical information or health condition of the subject user based on the request and a learned model which has learned pieces of physical information or health conditions of a plurality of second users and meal order histories of the plurality of second users; and an output means for outputting the response information.

In a meal recommendation method in accordance with an example aspect of the present invention: a computer receives physical information or a health condition of a subject user and a request pertaining to a meal menu; the computer generates response information including information pertaining to a meal menu which corresponds to the physical information or health condition of the subject user based on the request and a learned model which has learned pieces of physical information or health conditions of a plurality of second users and meal order histories of the plurality of second users; and the computer outputs the response information.

A meal recommendation program in accordance with an example aspect of the present invention causes a computer to carry out: a process of receiving physical information or a health condition of a subject user and a request pertaining to a meal menu; a process of generating response information including information pertaining to a meal menu which corresponds to the physical information or health condition of the subject user based on the request and a learned model which has learned pieces of physical information or health conditions of a plurality of second users and meal order histories of the plurality of second users; and a process of outputting the response information.

### Advantageous Effects of Invention

According to an example aspect of the present invention, it is possible to recommend a meal menu that is suitable for a user even if the user himself/herself does not recognize suitability.

### BRIEF DESCRIPTION OF DRAWINGS

FIG. 1 is a block diagram illustrating a configuration of a meal recommendation apparatus in accordance with a first example embodiment of the present invention.

FIG. 2 is a flowchart illustrating a flow of a meal recommendation method in accordance with the first example embodiment of the present invention.

FIG. 3 is a diagram illustrating learning of feature quantities in graph-based relationship learning.

FIG. 4 is a diagram illustrating an overview of a meal recommendation method in accordance with a second example embodiment of the present invention.

FIG. 5 is a block diagram illustrating a configuration of a meal recommendation apparatus in accordance with the second example embodiment of the present invention.

FIG. 6 is a flowchart illustrating a flow of a process carried out by the meal recommendation apparatus in accordance with the second example embodiment of the present invention.

FIG. 7 is a diagram illustrating an example of response information.

FIG. 8 is a diagram illustrating an overview of a meal recommendation method in accordance with a third example embodiment of the present invention.

FIG. 9 is a block diagram illustrating a configuration of a meal recommendation apparatus in accordance with the third example embodiment of the present invention.

FIG. 10 is a flowchart illustrating a flow of a process carried out by the meal recommendation apparatus in accordance with the third example embodiment of the present invention.

FIG. 11 is a diagram illustrating an overview of a meal recommendation method in accordance with a fourth example embodiment of the present invention.

FIG. 12 is a block diagram illustrating a configuration of a meal recommendation apparatus in accordance with the fourth example embodiment of the present invention.

FIG. 13 is a flowchart illustrating a flow of a process carried out by the meal recommendation apparatus in accordance with the fourth example embodiment of the present invention.

FIG. 14 is a diagram illustrating an example of predicting a property of a subject user based on a feature quantity calculated from a subject user graph and a second user graph.

FIG. 15 is a configuration diagram for realizing a meal recommendation apparatus by software.

### EXAMPLE EMBODIMENTS

### First Example Embodiment

The following description will discuss a first example embodiment of the present invention in detail, with reference to the drawings. The present example embodiment is a basic form of example embodiments described later.

### (Meal Recommendation Apparatus)

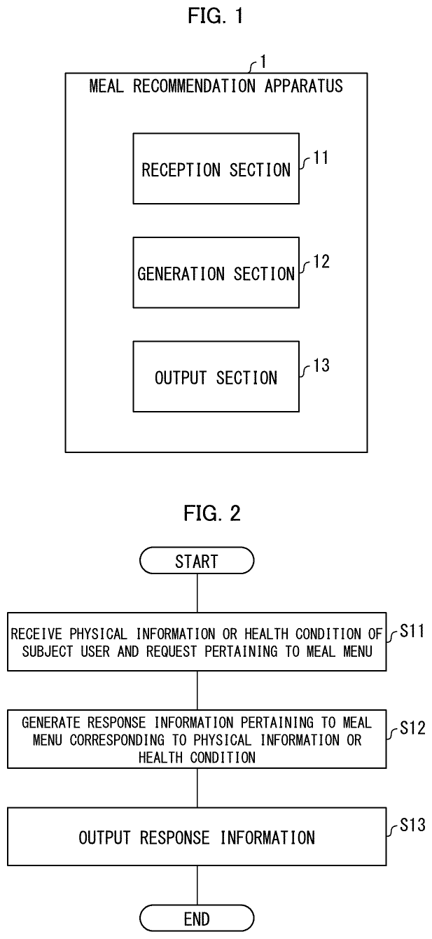

The following description will discuss a configuration of a meal recommendation apparatus 1 in accordance with the present example embodiment, with reference to the drawings. FIG. 1 is a block diagram illustrating the configuration of the meal recommendation apparatus 1 in accordance with the present example embodiment.

As illustrated in FIG. 1, the meal recommendation apparatus 1 includes a reception section (reception means) 11, a generation section (generation means) 12, and an output section (output means) 13.

The reception section 11 receives physical information or a health condition of a subject user and a request pertaining to a meal menu. Here, examples of the physical information of the subject user include, but not limited to, extension and a body weight of the user. For example, the physical information of the subject user may be configured to further include a body fat percentage, a body mass index (BMI), and the like of the user. The physical information may further include information indicating a date and time when the physical information was updated by body measurement or the like.

Examples of information (also simply referred to as a health condition) indicating a health condition of the subject user include, but not limited to, at least one selected from the group consisting of a blood pressure, a cholesterol value, a uric acid value, and allergy information of the user. The health condition of the subject user may further include a date and time when the health condition was updated by a medical checkup or the like. The request pertaining to a meal menu may include an order history of the subject user.

The generation section 12 generates response information including information pertaining to a meal menu which corresponds to the physical information or health condition of the subject user based on the request and a learned model pieces of physical information or health conditions of a plurality of second users and meal order histories of the plurality of second users. Note that the second user means, for example, each of persons other than the subject user, and a person who does not use the meal recommendation apparatus 1 can also be a second user. Here, examples of the meal order history of the subject user include, but not limited to, identification information of a product such as an ordered date and time, an ordered product name (meal name), a product ID (meal menu ID), and a price. The output section 13 outputs the response information generated by the generation section 12.

According to the meal recommendation apparatus 1 having the above configuration, it is possible to generate response information including information pertaining to a meal menu which corresponds to physical information or a health condition of the subject user based on a relation between pieces of physical information or health conditions of a plurality of second users and meal order histories of the plurality of second users, and present the generated response information to the subject user. Therefore, according to the above configuration, it is possible to bring about an example advantage of recommending a meal menu that is suitable for a user even if the user himself/herself does not recognize suitability.

### (Meal Recommendation Program)

The functions of the meal recommendation apparatus 1 described above can also be realized by a program. A meal recommendation program in accordance with the present example embodiment causes a computer to carry out: a process of receiving physical information or a health condition of a subject user and a request pertaining to a meal menu; a process of generating response information including information pertaining to a meal menu which corresponds to the physical information or health condition of the subject user based on the request and a learned model which has learned pieces of physical information or health conditions of a plurality of second users and meal order histories of the plurality of second users; and a process of outputting the response information. According to the meal recommendation program, it is possible to bring about an example advantage of recommending a meal menu that is suitable for a user even if the user himself/herself does not recognize suitability.

### (Meal Recommendation Method)

The following description will discuss a meal recommendation method in accordance with the present example embodiment, with reference to FIG. 2. FIG. 2 is a flowchart illustrating a flow of a meal recommendation method in accordance with the first example embodiment of the present invention.

In S11, a computer receives physical information or a health condition of a subject user and a request pertaining to a meal menu. The request may be received via an arbitrary input apparatus. For example, the request may be received via a mouse, a keyboard, a touch panel, or an audio input apparatus.

In S12, the computer generates response information including information pertaining to a meal menu which corresponds to the physical information or health condition of the subject user based on the request and a learned model which has learned pieces of physical information or health conditions of a plurality of second users and meal order histories of the plurality of second users.

In S13, the computer outputs the response information. The information may be output to an arbitrary apparatus. For example, the information may be output to a display apparatus so that the information is displayed, or the information may be output to an audio output apparatus so that the information is output as audio.

As described above, in the meal recommendation method in accordance with the present example embodiment: the computer receives physical information or a health condition of a subject user and a request pertaining to a meal menu (S11); the computer generates response information including information pertaining to a meal menu which corresponds to the physical information or health condition of the subject user based on the request and a learned model which has learned pieces of physical information or health conditions of a plurality of second users and meal order histories of the plurality of second users (S12); and the computer outputs the response information (S13). According to the meal recommendation method, it is possible to bring about an example advantage of recommending a meal menu that is suitable for a user even if the user himself/herself does not recognize suitability.

An execution subject of each step in the meal recommendation method described above may be a single computer (the meal recommendation apparatus 1), or execution subjects of respective steps may be different computers. The same applies to flows described later in the second and subsequent example embodiments.

### [Graph and Learning]

The following description will discuss a graph that is an example of information which can be used to recommend a meal in the first example embodiment and in the subsequent example embodiments (hereinafter, referred to as “each example embodiment”). The following description will also discuss learning of the graph and prediction using the graph.

### (Graph)

The graph herein refers to data having a structure composed of a plurality of nodes and links connecting the nodes. A type of link representing a relation between nodes is also called a “relation”. Alternatively, the link may be called an “edge”. The graph roughly includes a directed graph in which each link has directionality, and an undirected graph in which each link has no directionality. It is possible to utilize either a directed graph or an undirected graph. It is also possible to use those graphs in combination.

n a case of using a graph in each example embodiment, a node in the graph only needs to represent a tangible or intangible element pertaining to a person (user). For example, it is possible to utilize a graph including nodes representing various elements such as:
personal identification information such as a name and a personal ID;
age;
gender;
order history;
physical information; and
health condition.
   As described above, examples of the order history include, but not limited to, identification information of a product such as an ordered date and time, an ordered product name (meal name), a product ID (meal menu ID), and a price. Moreover, as described above, examples of physical information of a certain user include, but not limited to, extension and a body weight of the certain user. For example, the physical information of the certain user may be configured to further include a body fat percentage, a body mass index (BMI), and the like of the certain user. The physical information of the certain user may further include information indicating a date and time when the physical information was updated by body measurement or the like.

As described above, examples of information (also simply referred to as a health condition) indicating a health condition of a certain user include, but not limited to, at least one selected from the group consisting of a blood pressure, a cholesterol value, a uric acid value, and allergy information of the certain user. The health condition of the certain user may further include a date and time when the health condition was updated by a medical checkup or the like.

Note that identification information of a person (such as a name and personal ID), an age, and a gender may be referred to as attributes of the user. Moreover, the physical information and the health condition may be referred to as properties.

The graph may include a plurality of nodes corresponding to a single element. For example, as nodes indicating pieces of physical information, pieces of physical information of the user may be represented by respective individual nodes, such as a node indicating a body height and a node indicating a body weight. The same applies to other elements.

In a case where there are nodes corresponding to elements as described above, a link represents:
a relation between a certain element and a person;
a relation between a certain element and an order history;
a relation between a certain element and physical information;
a relation between a certain element and a health condition; or the like.
   For example, a link connecting a node indicating a health condition to a node indicating an order history may represent a relation in which the order history is a factor of the health condition.

Note that nodes in a graph in each example embodiment may include elements pertaining to a product, in addition to tangible or intangible elements pertaining to a person (user) as described above. For example, a graph including nodes representing, in addition to the above-described product identification information such as a product ID (meal menu ID), various elements as below can be utilized:
a product category;
store identification information such as a restaurant ID and a store ID;
location information of a restaurant or a store; and
foodstuffs for a product (meal menu).
   Here, the foodstuffs for a product (meal menu) include information of raw materials, an allergic substance, and the like used in the product. In a case where there are nodes corresponding to elements as described above, a link represents:
a relation between a certain product and a product category;
a relation between a certain product and a restaurant or store; or the like.
   For example, a link connecting a node indicating a product ID to a node indicating a restaurant ID may represent which restaurant has provided the product.

### (Learning and Prediction)

For a graph as described above, a machine learning technique can be applied to carry out graph-based relationship learning. Such learning makes it possible to carry out a classification process or a prediction process using a graph. In each example embodiment, such learning may be carried out as a part of meal menu recommendation, or a learned graph which has already been subjected to such learning may be used.

In graph-based relationship learning, first, a feature quantity of each node is calculated. The feature quantity may be calculated in the form of, for example, a feature quantity vector. By representing a feature quantity of each node by a feature quantity vector, it is possible to carry out learning also for a graph in which nodes of various forms mixedly exist. For example, graph-based relationship learning can also be carried out for a graph including an image, a numerical value, and the like indicating various elements as described above. For example, a photograph of a product can be regarded as a node.

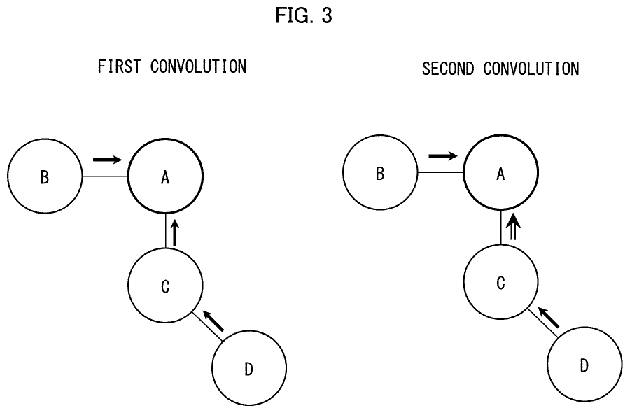

Next, the feature quantity of each node is updated based on a link connected to that node and a node which is a connection destination of the link. This process is similar to a convolution process in a convolutional neural network. This will be described with reference to FIG. 3. FIG. 3 is a diagram illustrating learning of feature quantities in graph-based relationship learning.

In a graph illustrated in FIG. 3, four nodes A through D are included. The nodes B and C are connected to the node A, and the node D is connected to the node C. After calculating initial feature quantities of these four nodes, a plurality of times of convolution are carried out as described below to update the feature quantities of the respective nodes.

In the first convolution, feature quantities of the nodes B and C connected to the node A are each multiplied by a predetermined weight and are then added to an initial feature quantity of the node A. For the node C, a feature quantity of the node D is multiplied by a predetermined weight and is then added to an initial feature quantity of the node C. Note that, in a case of an effective graph, the weight is adjusted according to a direction of the link.

In the second convolution, similarly to the first convolution, a feature quantity of a node linked to each node is multiplied by a predetermined weight, and is then added to a feature quantity of the each node. Here, the feature quantity of the node C reflects the feature quantity of the node D by the first convolution. Therefore, by the second convolution, not only the feature quantity of the node C but also the feature quantity of the node D are reflected in the node A.

By repeating the above-described process by a number of times corresponding to the hierarchy of nodes, feature quantities of nodes which are directly or indirectly connected to each other by links are mutually reflected. In graph-based relationship learning, a weight value used for the above-described weighting is optimized based on a known relationship between nodes. By using such a learned graph, it is possible to predict an inter-node relation and predict a link destination node, as described below.

### (Inter-Node Relation Prediction)

By carrying out the learning described above, it is possible to predict an inter-node relation which is not explicitly indicated in the original graph. In a case of carrying out inter-node relation prediction, a user may specify two nodes and make a request for returning a relation between those nodes. For example, in a case where a request of inquiring about a relation between a node of “user A” and a node of “user B” is input from a user, it is possible to predict, by inter-node relation prediction, that a relation (i.e., a link) that connects these nodes is “similar”. In the inter-node relation prediction, it is possible to calculate a probability (likelihood) of a prediction result. The same applies to node prediction described below.

### (Node Prediction)

By carrying out the above-described learning, it is also possible to predict a node that is connected to a certain node by a predetermined link. In a case of carrying out node prediction, a user may specify a single node and a link starting from that single node, and make a request for returning a link destination node. For example, it is assumed that a request for inquiring about a node connected to a node of “user A” by a link of “health condition” is input from a user. In this case, for example, it is possible to predict, by node prediction, whether a node connected to the node of “user A” by the link of “health condition” is “good” or “tendency toward obesity”.

### Second Example Embodiment

### (Overview)

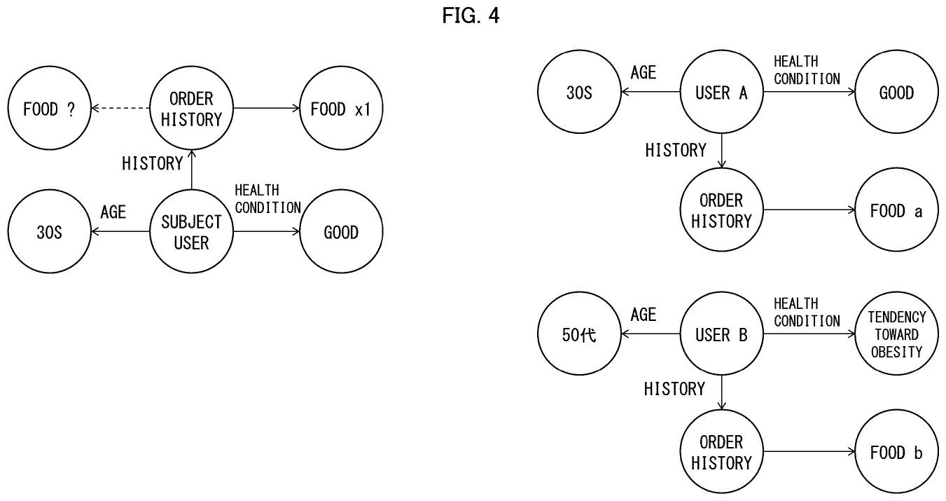

FIG. 4 is a diagram illustrating an overview of a meal recommendation method in accordance with the present example embodiment. In the present example embodiment, an example will be described in which a subject user graph and a second user graph are used to recommend a meal.

The second user graph is a learned model and is a graph which includes (i) a node indicating a second user (which is synonymous with “second user” in the first example embodiment) who is different from a subject user who is a subject to whom a meal is recommended, (ii) nodes each indicating physical information, a health condition, or a meal menu related to the second user, and (iii) links indicating relationships between the nodes, and which has learned the relationships between the nodes. The second user graph can also be called a knowledge graph. A set of nodes and links corresponding to a single second user may be referred to as a second user graph, or a set of nodes and links corresponding to a plurality of second users may be collectively referred to as a second user graph.

For example, in FIG. 4, a graph including a node of “user A” and a graph including a node of “user B” are second user graphs. The second user graph pertaining to the user A includes: a node indicating that an age of the user A is “30s”; a node indicating that a health condition of the user A is “good”; a node indicating an “order history” as a history of the user A; and a node of “food a” linked to the node indicating the “order history”.

Similarly, the second user graph pertaining to the user B includes: a node indicating that an age of the user B is “50s”; a node indicating that a health condition of the user B is “tendency toward obesity”; a node indicating an “order history” as a history of the user B; and a node of “food b” linked to the node indicating the “order history”.

The second user graph illustrated in FIG. 4 is merely an example, and the second user graph may include a node indicating physical information in addition to or in place of the node indicating the health condition described above.

As such, the second user graph illustrated in FIG. 4 has learned, for each of a plurality of second users, a relation between physical information, a health condition, and a meal menu.

The second user graph may include nodes each indicating past physical information, a past health condition, or a past meal menu related to the subject user, and links indicating relationships between the nodes. For example, the second user graph may be a graph which includes (i) nodes each indicating past physical information, a past health condition, or a past meal menu related to the subject user, (ii) nodes each indicating physical information, a health condition, or a meal menu related to the second user, and (iii) links indicating relationships between the nodes, and which has learned the relationships between the nodes.

The subject user graph is a graph including a plurality of nodes pertaining to a subject user who is a subject to whom a meal is recommended. In FIG. 4, a graph including a node “subject user” is a subject user graph. The subject user graph includes: a node indicating that an age of the subject user is “30s”; a node indicating that a health condition of the subject user is “good”; a node indicating an “order history” as a history of the subject user; and a node of “food x1” linked to the node indicating the “order history”. Such a subject user graph can be generated from various kinds of information pertaining to the subject user.

For example, it is possible to generate a subject user graph by receiving, from a subject user, various kinds of information including an age and a health condition of the subject user and a request including an order history, and referring to the received various kinds of information and request. At this time, the request may include a category of a meal menu which the subject user wants, and the like. In this case, for example, a graph having a node pertaining to a meal menu that conforms to the category among second user graphs is a subject of link prediction.

The subject graph illustrated in FIG. 4 is merely an example, and the subject graph may include a node indicating physical information in addition to or in place of the node indicating the health condition described above.

By training the second user graph as described above, it is possible to carry out link prediction for predicting what meal menu is suitable for what physical information or health condition. That is, in the meal recommendation method in accordance with the present example embodiment, a meal menu to be recommended to a subject user is predicted by link prediction, and response information including information pertaining to the predicted meal menu is generated and output.

For example, in the example of FIG. 4, link prediction may be carried out for predicting which node among nodes that are included in the second user graph and each indicate physical information, a health condition, or a meal menu related to the second user is likely to be connected to a node included in the subject user graph. Then, it is possible to generate and output response information including information pertaining to a meal menu corresponding to the predicted node.

### (Apparatus Configuration)

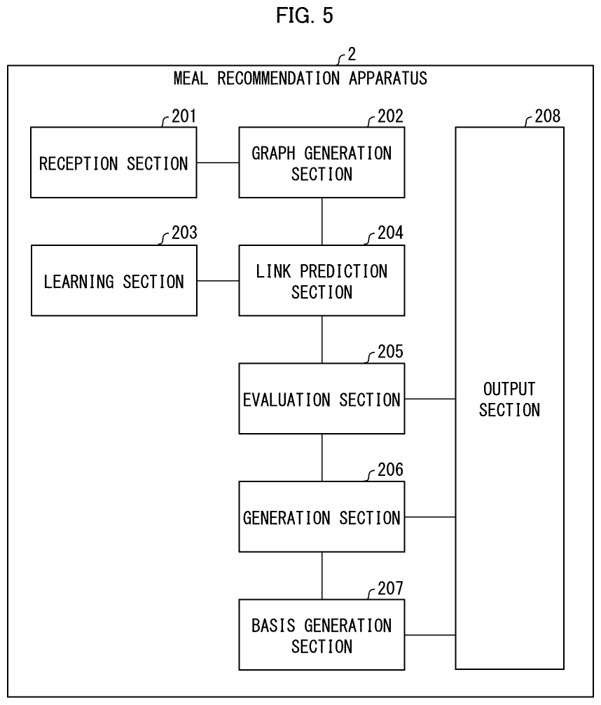

The following description will discuss a configuration of a meal recommendation apparatus 2 in accordance with the second example embodiment of the present invention, with reference to FIG. 5. FIG. 5 is a block diagram illustrating the configuration of the meal recommendation apparatus 2 in accordance with the present example embodiment.

As illustrated in FIG. 5, the meal recommendation apparatus 2 includes a reception section 201, a graph generation section 202, a learning section 203, a link prediction section 204, an evaluation section 205, a generation section 206, a basis generation section 207, and an output section 208.

In addition to these constituent elements, the meal recommendation apparatus 2 may include an input apparatus for receiving an input operation by a user, an output apparatus for outputting data output by the meal recommendation apparatus 2, a communication apparatus for enabling the meal recommendation apparatus 2 to communicate with another apparatus, and the like. An output mode of the output apparatus may be arbitrarily set, and may be, for example, display output or audio output.

The reception section 201 receives an age, physical information, or a health condition of a subject user and a request pertaining to a meal menu. Here, the request can include an order history of the subject user. The request may include information indicating a category of a meal menu (product), a restaurant name, or a store name which the subject user wishes, and the like.

The reception section 201 may receive input of a condition for a second user graph. The received condition is a condition that defines a restriction at the time when link prediction is carried out.

The graph generation section 202 generates a subject user graph representing the subject user in the form of graph based on an age, physical information, or a health condition of the subject user and a request pertaining to a meal menu. For example, the graph generation section 202 generates a subject user graph representing the subject user with a node indicating the subject user, a node indicating an age of the subject user, a node indicating a health condition of the subject user, a node indicating an order history of the subject user, and edges representing relationships between the nodes. Here, the subject user graph may include a node indicating physical information of the subject user in addition to a node indicating a health condition of the subject user or in place of the node indicating a health condition of the subject user.

The information pertaining to the subject user may be included in various kinds of information and a request received by the reception section 201 or may be acquired from a database in which pieces of information pertaining to a plurality of users are accumulated, or the like.

The learning section 203 learns, based on various kinds of information pertaining to a second user, a relationship between nodes included in a second user graph, in other words, a relation between an age, a health condition or physical information, and an order history, and generates a learned second user graph. Unless otherwise specified, the second user graph refers to a graph which has been subjected to learning by the learning section 203. The learned second user graph may be read into the meal recommendation apparatus 2. In such a case, the learning section 203 may be omitted.

The link prediction section 204 predicts, by link prediction using the subject user graph described above and the second user graph, a node which links to a node included in the subject user graph from among nodes which are included in the second user graph and indicate meal menus related to the second user, the link prediction being carried out for predicting a relationship between nodes which are not connected to each other by a link in the subject user graph and the second user graph.

In a configuration in which the reception section 201 receives input of a condition for the second user graph, the link prediction section 204 may predict, from among nodes that indicate meal menus and are included in a second user graph satisfying the condition, a node which links to a node included in the subject user graph.

The evaluation section 205 evaluates, based on another node included in the second user graph including the node predicted by the link prediction section 204, a recommendation level, for the subject user, of a meal menu indicated by the node. The evaluation method will be described later.

The generation section 206 generates response information including information pertaining to a meal menu which corresponds to the physical information or health condition of the subject user based on the request and a learned model which has learned pieces of physical information or health conditions of a plurality of second users and meal order histories of the plurality of second users. More specifically, the generation section 206 generates response information including information pertaining to a meal menu corresponding to a node that has been predicted by the link prediction section 204. As described above, the link prediction section 204 carries out link prediction using a second user graph. Therefore, the generation section 206 generates response information based on a result of link prediction by the link prediction section 204, and thereby generates response information based on a learned model.

The basis generation section 207 generates basis information indicating validity of response information generated by the generation section 206. The method for generating basis information will be described later.

The output section 208 outputs various pieces of information generated by the meal recommendation apparatus 2. For example, the output section 208 outputs response information generated by the generation section 206 and basis information indicated by the basis generation section 207. An output destination of the information may be arbitrarily set. For example, in a case where the meal recommendation apparatus 2 includes an output apparatus as described above, the information may be output to the output apparatus. Alternatively, for example, the information may be output to an output apparatus provided outside the meal recommendation apparatus 2.

As described above, the learned model used by the meal recommendation apparatus 2 may be a second user graph that is a graph which includes (i) a node indicating a second user, who is different from the subject user, (ii) nodes each indicating physical information, a health condition, or a meal menu related to the second user, and (iii) links indicating relationships between the nodes, and which has learned the relationships between the nodes. According to the configuration, it is possible to generate and output response information including information pertaining to a meal menu that corresponds to physical information or a health condition of the subject user, while taking into consideration a mutual relationship between physical information, a health condition, a meal menu, and the like of the second user.

As described above, the meal recommendation apparatus 2 may include the link prediction section 204 that predicts, by link prediction using a subject user graph and the second user graph, a node which links to a node included in the subject user graph from among nodes which are included in the second user graph and indicate meal menus related to the second user, the subject user graph including a plurality of nodes pertaining to the subject user, and the link prediction being carried out for predicting a relationship between nodes which are not connected to each other by a link in the subject user graph and the second user graph. The generation section 206 may generate response information including information pertaining to a meal menu corresponding to a node that has been predicted by the link prediction section 204.

A node which indicates physical information, a health condition, or a meal menu related to a second user and which links to a node included in the subject user graph may indicate physical information, a health condition, or a meal menu related to a meal menu that is suitable for the subject user. Therefore, according to the configuration, it is possible to recommend, to the subject user, a meal menu that is suitable for the subject user.

As described above, in the meal recommendation apparatus 2, the reception section 201 may receive input of a condition for the second user graph, and the link prediction section 204 may predict, from among nodes that indicate meal menus and are included in a second user graph satisfying the condition, a node which links to a node included in the subject user graph.

According to the configuration, a prediction range of a node that links to the subject user graph is narrowed down to a second user graph that satisfies the input condition. Thus, it is possible to predict a node conforming to an intention of the subject user. For example, in a case where the subject user has input “successful in weight reduction” as a condition, a node which links to a node included in the subject user graph is predicted from a second user graph which indicates that weight reduction has succeeded. In this case, it is possible to recommend, to the subject user, a meal menu that is more likely to lead to success of weight reduction.

### (Evaluation of Recommendation Level)

As described above, the evaluation section 205 evaluates, based on another node included in the second user graph including the node predicted by the link prediction section 204, a recommendation level, for the subject user, of a meal menu indicated by the node. The following description will discuss evaluation by the evaluation section 205.

A node that links to the subject user graph may indicate a meal menu that is suitable for the subject user. Then, each of nodes included in a second user graph including that node may indicate a factor that affects a recommendation level of the meal menu. For example, it is assumed that a node indicating that a health condition of a second user is good is included in a second user graph. In this case, a meal menu indicated by a node that is included in the second user graph and links to the subject user graph may contribute to improvement of the health condition of the subject user. Therefore, it can be said that a recommendation level of that meal menu is high.

According to the configuration, based on another node included in the second user graph including the node predicted by the link prediction means, a recommendation level, for the subject user, of a meal menu indicated by the node is evaluated. An evaluation method may be determined in advance based on a target node or the like. Then, the subject user may decide, according to the evaluation, whether to purchase the meal menu (order the meal). Thus, it is possible to contribute to purchase of a meal menu which is more favorable for the subject user.

Various criteria for evaluation can be applied. For example, evaluation can be carried out on the basis of a degree of conformity to a request. For example, it is assumed that a request includes a category of a meal menu which the subject user wants. In this case, the evaluation section 205 may set evaluation of a meal menu corresponding to a second user graph which includes a node indicating that category to be higher than evaluation of a meal menu corresponding to a second user graph which does not include a node indicating that category.

The evaluation section 205 may express an evaluation result by a numerical value. In the present example embodiment, an example is described in which the evaluation section 205 calculates a recommendation level, which is a numerical value indicating suitability of a meal menu for the subject user. In this case, for example, by setting, as a rule, a relation between a node included in a second user graph and a recommendation level in advance, the evaluation section 205 can calculate a recommendation level of each meal menu according to the rule.

The evaluation section 205 may evaluate a recommendation level of a meal menu with reference to a node indicating allergy information included in the subject user graph and a node indicating an allergic substance that links to a node indicating the meal menu included in the second user graph. For example, evaluation of a meal menu including an allergic substance that causes allergy in the subject user graph may be set to be lower than evaluation of a meal menu that does not include the allergic substance.

### (Method of Generating Basis Information)

As described above, the basis generation section 207 generates basis information indicating validity of response information generated by the generation section 206. For example, the basis generation section 207 may generate basis information including information pertaining to a person whose physical information or health condition is similar to that of the subject user, among a plurality of second users indicated in a second user graph. This configuration allows a user to refer to response information while taking into consideration basis information, and to precisely determine validity of the response information.

Various techniques can be applied as a method of generating basis information. For example, the basis generation section 207 may use, as basis information, a whole or a part of a second user graph including a node predicted by the link prediction section 204. For example, the basis generation section 207 may search for a second user graph which includes a node predicted by the link prediction section 204 and includes a predetermined number or more of nodes common to a node indicating physical information or a health condition of the subject user. Then, the basis generation section 207 may use, as basis information, a whole or a part of the second user graph detected by the search.

(Basis Generation with Respect to Link Prediction Result)

The basis generation section 207 can generate basis information also by analyzing a subject user graph and a second user graph. The following description will discuss a method of generating basis information by analyzing a subject user graph and a second user graph.

For example, the basis generation section 207 may mine one or more rules from the subject user graph and the second user graph using principal component analysis (PCA) reliability based on open-world assumption (OWA). The basis generation section 207 may generate basis information using one or more rules that have been mined. For example, a method described in the following literature can be applied to mining of a rule.

Luis Galarraga et. al, “Fast rule mining in ontological knowledge bases with AMIE+”, The VLDB Journal (2015) 24:707-730

For example, a rule to be processed by the basis generation section 207 is expressed by using Head r(x, y) and Body {B1, . . . , Bn}.

$${B}_{1} \hat{} {B}_{2} \hat{} ⋯ \hat{} {B}_{n} ⇒ r ( x , y )$$

This rule may also be expressed as

[symbol]

⇒r(x,y)

using a vector representation. Here, Head r(x, y) is also called an atom.

As a condition of the mining process, the basis generation section 207 uses the following conditions to carry out the mining process:
Connected: all values (variables, entities) in a rule are shared between different atoms;
Closed: all variables in a rule appear two times or more; and
Not reflexive: a rule containing a reflective atom (such as r(x, x)) is not mined.

The basis generation section 207 may use a head coverage (hc) defined by

$$hc ( \vec{B} ⇒ r ( x , y ) ) := \frac{supp ( \vec{B} ⇒ r ( x , y ) )}{size ( r )}$$

and PCA reliability defined by

$${conf}_{\text{?}} ( \vec{B} ⇒ r ( x , y ) ) := \frac{supp ( \vec{B} ⇒ r ( x , y ) )}{\# ( x , y ) : ∃ {z}_{1} , … , {z}_{m} , {y}^{′} \text{:} \vec{B} \hat{} r ( x , {y}^{′} )}$$

$$\text{?} \text{indicates text missing or illegible when filed}$$

to carry out the mining process. By using PCA reliability, it is possible to mine a rule with higher accuracy, as compared with a case of using standard reliability. Therefore, by using the above configuration, it is possible for the basis generation section 207 to generate highly reliable basis information.

For example, it is assumed that the basis generation section 207 has mined a rule that, when “a food a is included in an order history of a certain person”, “a health condition of the certain person is good”. In this case, if the link prediction section 204 has predicted a node indicating the food a, the basis generation section 207 may generate, as a basis of the prediction, basis information indicating that there is a tendency that “a health condition of a person whose order history includes the food a is good”.

### (Flow of Process)

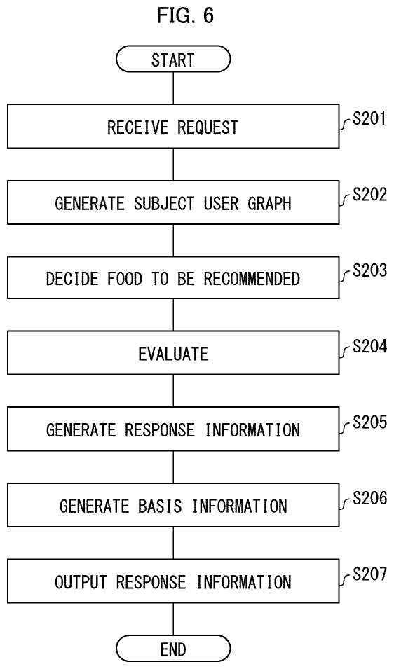

The following description will discuss a flow of a process (meal recommendation method) carried out by the meal recommendation apparatus 2, with reference to FIG. 6. FIG. 6 is a flowchart illustrating the flow of the process carried out by the meal recommendation apparatus 2.

In S201, the reception section 201 receives an age, physical information, or a health condition of a subject user and a request pertaining to a meal menu. Here, the request can include an order history of the subject user. The request may include information indicating a category of a meal menu (product), a restaurant name, or a store name which the subject user wishes, and the like.

Subsequently, in S202, the graph generation section 202 generates a subject user graph based on the information input in S201.

In S203, the link prediction section 204 predicts, by link prediction using the subject user graph generated in S202 and the second user graph, a node which links to a node included in the subject user graph from among nodes which are included in the second user graph and indicate meal menus related to the second user, the link prediction being carried out for predicting a relationship between nodes which are not connected to each other by a link in the subject user graph and the second user graph. The link prediction section 204 may predict a plurality of nodes. In this case, the basis generation section 207 may generate basis information indicating a basis of a prediction result by the link prediction section 204 by analyzing the subject user graph and the second user graph.

In S204, the evaluation section 205 evaluates, based on another node included in the second user graph including the node predicted in S203, a recommendation level, for the subject user, of a meal menu indicated by the node. For example, the evaluation section 205 may evaluate a recommendation level of a meal menu based on a degree of conformity to the request received in S201. In a case where a plurality of nodes are predicted in S203, the evaluation section 205 carries out evaluation for each of the predicted nodes.

In S205, the generation section 206 infers a meal menu corresponding to the node predicted in S203, and generates response information including information pertaining to the inferred meal menu. As described above, the second user graph is a graph which includes (i) a node indicating a second user, who is different from the subject user, (ii) nodes each indicating physical information, a health condition, or a meal menu related to the second user, and (iii) links indicating relationships between the nodes, and which has learned the relationships between the nodes. Then, by link prediction using the subject user graph and the second user graph, a node is predicted in S203. Therefore, it can be said that, in S205, response information including information pertaining to a meal menu which corresponds to physical information or a health condition of the subject user is generated based on the request and the health condition of the subject user using a learned model which has learned pieces of physical information or health conditions of a plurality of second users and meal order histories of the plurality of second users.

For example, the generation section 206 may generate response information indicating a meal menu of which an evaluation result in S204 is up to a predetermined ordinal number among meal menus corresponding to the node predicted in S203. For example, the generation section 206 may generate response information indicating, among meal menus corresponding to the node predicted in S203, a meal menu that conforms to the request received in S201. Besides the above examples, the generation section 206 may generate response information indicating, for example, a meal menu corresponding to the node predicted in S203 and an evaluation result in S204.

In S206, the basis generation section 207 generates basis information indicating validity of the response information generated in S205. For example, the basis generation section 207 may generate basis information including information pertaining to a person whose physical information or health condition is similar to that of the subject user, among a plurality of second users indicated in a second user graph.

In S207, the output section 208 outputs the response information which has been generated in S206. In this case, the output section 208 may output the basis information which has been generated in S206. Thus, the process of FIG. 6 ends.

### (Example of Response Information)

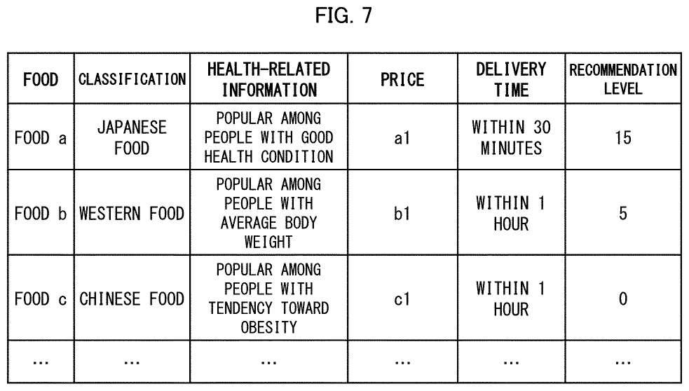

In S207, response information as illustrated in FIG. 7 for example may be output. FIG. 7 is a diagram illustrating an example of response information. The response information illustrated in FIG. 7 includes six items in total, i.e., “food”, “classification”, “health-related information”, “price”, “delivery time”, and “recommendation level”. The response information may be configured to further include allergy information identified from a second user graph.

The “food” is a meal menu inferred by the generation section 206 according to a node predicted by the link prediction section 204. In the example of FIG. 7, foods a through c are included as meal menus. The “classification” indicates a category of a meal menu. The “health-related information” is information pertaining to a relevance between a meal menu and a health condition. The “price” is a price of a meal menu. The “delivery time” is an estimated delivery time at the time when a meal menu is ordered. The “recommendation level” indicates an evaluation result by the evaluation section 205 with respect to a meal menu. Note that the “delivery time” can be estimated by the generation section 206 from, for example, location information linked to the meal menu inferred by the generation section 206 and location information of the subject user.

Among those items, “classification”, “health-related information”, “price”, and “delivery time” can be identified from a second user graph. The basis generation section 207 may generate such information as basis information.

The “recommendation level” may be calculated by the evaluation section 205 based on various kinds of information identified from a second user graph. For example, the recommendation level may be calculated so that evaluation of a meal menu corresponding to a second user graph including a node indicating a better health condition is higher than evaluation of a meal menu corresponding to a second user graph including a node indicating the other health condition.

In the example of FIG. 7, recommendation levels of foods a through c are 15, 5, and 0, respectively. For example, in a case where a rule as described below has been set in advance, the evaluation section 205 can calculate a recommendation level of each meal menu according to the rule. Such a rule may be as follows: if a meal menu corresponds to a second user graph including a node indicating a good health condition, the recommendation level is +15; and if a meal menu corresponds to a second user graph including a node indicating an average health condition, the recommendation level is +5.

In the meal recommendation apparatus 2 configured as described above, the link prediction section 204 carries out link prediction using the subject user graph and the second user graph. By carrying out such link prediction, the following example advantages are brought about.

(1) Utilization of multi-stage relation: A recommendation process can be carried out also for a case where a first user, who is a friend of a second user who has purchased a product similar to a product purchased by a third user, has purchased another product.

(2) Explanatory property: A recommendation reason can be explained using a rule in a graph (i.e., a rule supported by a certain number of relations in the graph).

### Third Example Embodiment

### (Overview)

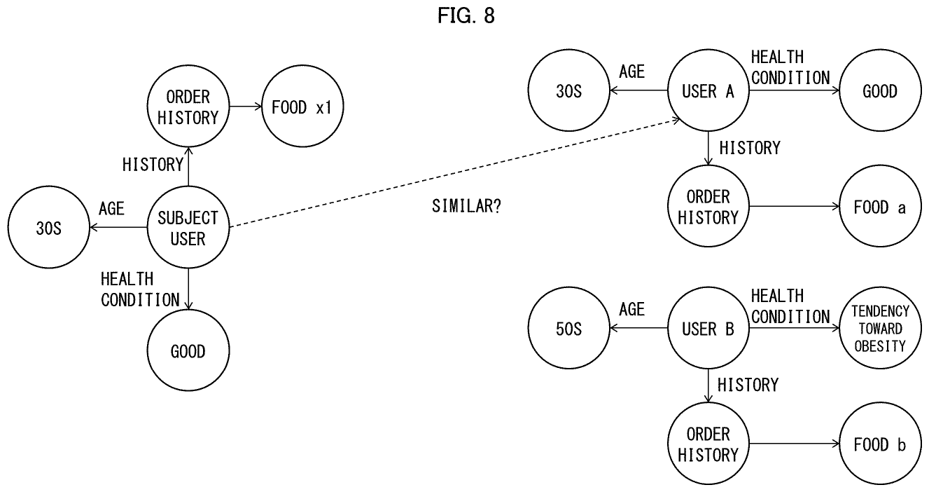

FIG. 8 is a diagram illustrating an overview of a meal recommendation method in accordance with the present example embodiment. In the present example embodiment, an example will be described in which a subject user graph including a plurality of nodes pertaining to a subject user and a plurality of second user graphs respectively generated for a plurality of second users are used to recommend a meal menu.

In the meal recommendation method in accordance with the present example embodiment, as in the first and second example embodiments, physical information or a health condition of a subject user and a request pertaining to a meal menu are received. Here, the request pertaining to a meal menu may include an order history of the subject user.

Next, in the meal recommendation method in accordance with the present example embodiment, a subject user graph is generated based on physical information or a health condition of the subject user and a request pertaining to a meal menu. In the example of FIG. 8, the subject user graph is a graph in which a node of “order history” is connected to a node of “subject user” by a link of “history”, a node of “30s” is connected to the node of “subject user” by a link of “age”, and a node of “good” is connected to the node of “subject user” by a link of “health condition”.

In the meal recommendation method in accordance with the present example embodiment, a second user having a predetermined relationship with the subject user is identified by link prediction using the subject user graph generated as described above and the second user graphs generated for the respective plurality of second users. The second user graphs to be used are generated for the respective plurality of second users, and have learned the predetermined relationship between the plurality of second users.

In the example of FIG. 8, a second user (hereinafter, referred to also as a similar user) similar to the subject user is predicted by link prediction using second user graphs including a node of a user A and a node of a user B. In this example, the learning is carried out in such a manner that dissimilar second users are not connected by a link “similar” (i.e., dissimilarity is regarded as a negative example). However, a link “dissimilar” may be learned.

Information pertaining to a similar user of a subject user for whom recommendation is carried out is useful for carrying out recommendation suitable for the subject user. Therefore, according to the configuration, it is possible to carry out recommendation suitable for the subject user.

It is also possible to carry out evaluation of a similar user predicted as described above, and recommend a meal menu according to the evaluation result. For example, a meal menu can be recommended by the following process.
A second user whose physical information and product category in an order history are similar to those of the subject user is predicted as a similar user.
Among a plurality of similar users who have been predicted, a similar user whose health condition has been updated within a predetermined period (e.g., within recent 6 months) and whose health condition is good is extracted as a similar user with high evaluation.
Among an order history of the extracted similar user, a meal menu which has been ordered frequently (e.g., the order frequency thereof is within the top three) is extracted, and the meal menu is recommended.

### (Apparatus Configuration)

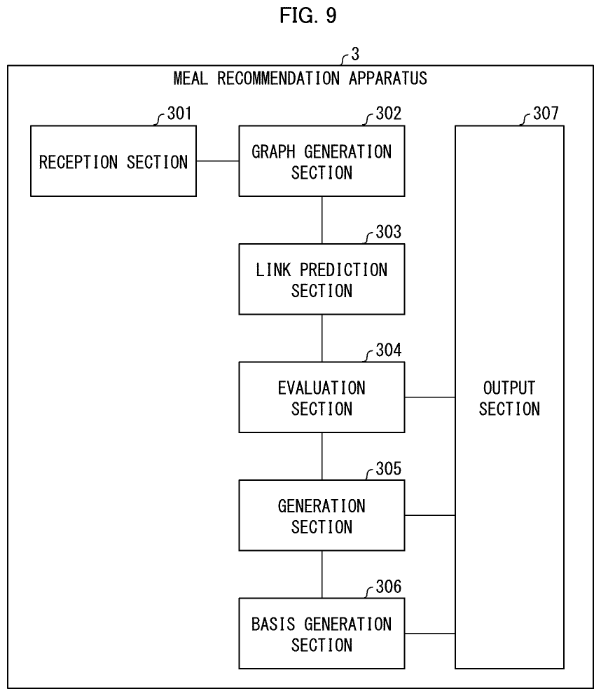

The following description will discuss a configuration of a meal recommendation apparatus 3 in accordance with the third example embodiment of the present invention, with reference to FIG. 9. FIG. 9 is a block diagram illustrating the configuration of the meal recommendation apparatus 3 in accordance with the present example embodiment.

As illustrated in FIG. 9, the meal recommendation apparatus 3 includes a reception section 301, a graph generation section 302, a link prediction section 303, an evaluation section 304, a generation section 305, a basis generation section 306, and an output section 307. As with the meal recommendation apparatus 2 in accordance with the second example embodiment, the meal recommendation apparatus 3 may include, in addition to those constituent elements, a learning section, an input apparatus, an output apparatus, a communication apparatus, and the like.

The reception section 301 receives an age, physical information, or a health condition of a subject user and a request pertaining to a meal menu. Here, the request can include an order history of the subject user. The request may include information indicating a category of a meal menu (product), a restaurant name, or a store name which the subject user wishes, and the like.

The graph generation section 302 generates a subject user graph based on an age, physical information, or a health condition of the subject user and a request pertaining to a meal menu. For example, the graph generation section 202 generates a subject user graph representing the subject user with a node indicating the subject user, a node indicating an age of the subject user, a node indicating a health condition of the subject user, a node indicating an order history of the subject user, and edges representing relationships between the nodes. Here, the subject user graph may include, in addition to a node indicating a health condition of the subject user or in place of a node indicating a health condition of the subject user, a node indicating physical information of the subject user or a node pertaining to the meal menu which has been requested by the subject user.

The link prediction section 303 identifies a second user who has a predetermined relationship with the subject user by link prediction using a subject user graph and a plurality of second user graphs, the subject user graph including a plurality of nodes pertaining to the subject user, the plurality of second user graphs having been respectively generated for a plurality of second users, and the link prediction being carried out for predicting a relationship between nodes which are not connected to each other by a link in the subject user graph and the plurality of second user graphs. The predetermined relationship may be a relationship of being similar as in the example of FIG. 8, or may be another relationship. For example, the link prediction section 303 may identify a second user who is not similar to the subject user, or can identify a second user who belongs to the same age zone as the subject user, a second user who has commonality in physical information with the subject user, or the like.

The evaluation section 304 evaluates the second user predicted by the link prediction section 303, i.e., the second user who has a predetermined relationship with the subject user. For example, in a case where the link prediction section 303 has predicted a similar user, the evaluation section 304 may calculate a higher recommendation level if a node indicating a better health condition is included in a second user graph of the similar user.

The evaluation section 304 may evaluate, based on a node included in a second user graph of the second user predicted by the link prediction section 303, i.e., the second user having a predetermined relationship with the subject user, a recommendation level, for the subject user, of a meal menu indicated by the node.

The generation section 305 generates the response information including information pertaining to a meal menu related to the second user who has been identified by the link prediction section 303. For example, the generation section 305 may generate response information that recommends, to the subject user, a meal menu indicated by a node included in a second user graph of the second user who has been identified by the link prediction section 303. As described above, the link prediction section 303 carries out link prediction using the second user graphs each of which is a learned model. Therefore, the generation section 305 generates response information based on a result of link prediction by the link prediction section 303, and thereby generates response information based on the learned model.

The generation section 305 may generate response information corresponding to an evaluation result by the evaluation section 304. For example, in a case where the link prediction section 303 has predicted a similar user, the evaluation section 304 may calculate a higher recommendation level if a node indicating a better health condition is included in a second user graph of the similar user. Then, the generation section 305 may generate response information for recommending a meal menu for which the recommendation level calculated by the evaluation section 304 is equal to or more than a predetermined value.

The basis generation section 306 generates basis information indicating validity of response information generated by the generation section 305. For example, the basis generation section 306 may generate basis information including information pertaining to a health condition included in the second user graph of the similar user predicted by the link prediction section 303. This configuration allows a user to refer to response information while taking into consideration a basis thereof, and to precisely determine validity of the meal menu. The basis generation section 306 may generate basis information pertaining to a result of link prediction by the link prediction section 303 by analyzing the subject user graph and the second user graphs.

The output section 307 outputs, for example, response information generated by the generation section 305. An output destination of the information is not particularly limited, as with the output section 208 in accordance with the second example embodiment.

As described above, the meal recommendation apparatus 3 includes the link prediction section 303 which identifies a second user who has a predetermined relationship with the subject user by link prediction using a subject user graph and a plurality of second user graphs, the subject user graph including a plurality of nodes pertaining to the subject user, the plurality of second user graphs having been respectively generated for a plurality of second users, and the link prediction being carried out for predicting a relationship between nodes which are not connected to each other by a link in the subject user graph and the plurality of second user graphs. Then, the generation section 305 generates the response information including information pertaining to a meal menu related to the second user who has been identified by the link prediction section 303. Information pertaining to a second user having a predetermined relationship with the subject user for whom recommendation is carried out is useful for carrying out recommendation suitable for the subject user. Therefore, according to the configuration, it is possible to carry out recommendation suitable for the subject user.

As described above, the link prediction section 303 may identify a second user who is similar to the subject user; and the generation section 305 may generate response information that recommends, to the subject user, a meal menu indicated by a node included in a second user graph of the second user who has been identified by the link prediction section 303. A meal menu related to a second user similar to the subject user is more likely to be suitable for the subject user. Therefore, according to the configuration, it is possible to recommend, to the user, a meal menu that is more likely to be suitable for the subject user.

### (Flow of Process)

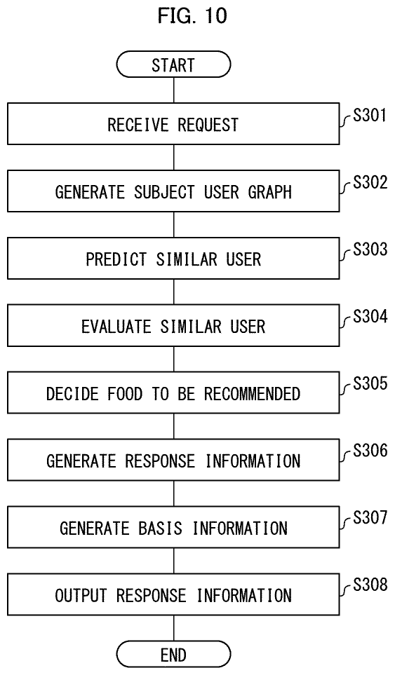

The following description will discuss a flow of a process (meal recommendation method) carried out by the meal recommendation apparatus 3, with reference to FIG. 10. FIG. 10 is a flowchart illustrating the flow of the process carried out by the meal recommendation apparatus 3.

In S301, the reception section 301 receives an age, physical information, or a health condition of a subject user and a request pertaining to a meal menu. Here, the request can include an order history of the subject user. The request may include information indicating a category of a meal menu (product), a restaurant name, or a store name which the subject user wishes, and the like.

Subsequently, in S302, the graph generation section 302 generates a subject user graph based on the information input in S301.

In S303, the link prediction section 303 predicts a similar user who is similar to the subject user by link prediction using the subject user graph generated in S302 and a plurality of second user graphs which have been respectively generated for a plurality of second users, the link prediction being carried out for predicting a relationship between nodes which are not connected to each other by a link in the subject user graph and the second user graphs.

In S304, the evaluation section 304 evaluates the similar user predicted in S303. For example, the evaluation section 304 may calculate a higher recommendation level if a node indicating a better health condition is included in a second user graph of the similar user. The evaluation section 304 may carry out the above evaluation on the basis of a degree of conformity to the request received in S301. In a case where a plurality of similar users are predicted in S303, the evaluation section 304 carries out evaluation for each of the predicted similar users.

In S305, the generation section 305 identifies a meal menu indicated by a node included in a second user graph of the similar user predicted in S303. That is, a food to be recommended to the subject user is decided. The generation section 305 generates response information including information pertaining to the identified meal menu. As described above, the second user graph is a graph which includes (i) a node indicating a second user, who is different from the subject user, (ii) nodes each indicating physical information, a health condition, or a meal menu related to the second user, and (iii) links indicating relationships between the nodes, and which has learned the relationships between the nodes. Then, in S303, a similar user is identified by link prediction using the subject user graph and the second user graphs. Therefore, it can be said that, in S305, response information including information pertaining to a meal menu which corresponds to physical information or a health condition of the subject user is generated based on the request and the health condition of the subject user using a learned model which has learned pieces of physical information or health conditions of a plurality of second users and meal order histories of the plurality of second users.

For example, the generation section 305 may generate response information indicating a meal menu indicated by a node included in a second user graph of the similar user of whom an evaluation result in S304 is up to a predetermined ordinal number among meal menus indicated by nodes included in second user graphs of similar users predicted in S303. For example, the generation section 305 may generate response information indicating, among meal menus indicated by nodes included in the second user graph of the similar user predicted in S303, a meal menu that conforms to the request received in S301. Besides the above examples, for example, the generation section 305 may generate response information indicating a meal menu indicated by a node included in the second user graph of the similar user predicted in S303 and an evaluation result in S304.

In S306, the basis generation section 306 generates basis information indicating validity of the response information generated in S306. For example, the basis generation section 306 may generate basis information including information pertaining to a health condition included in the second user graph of the similar user predicted in S303.

In S307, the output section 307 outputs the response information which has been generated in S306. At this time, the output section 208 may output also the basis information which has been generated in S306. Thus, the process of FIG. 10 ends.

### Fourth Example Embodiment

### (Overview)

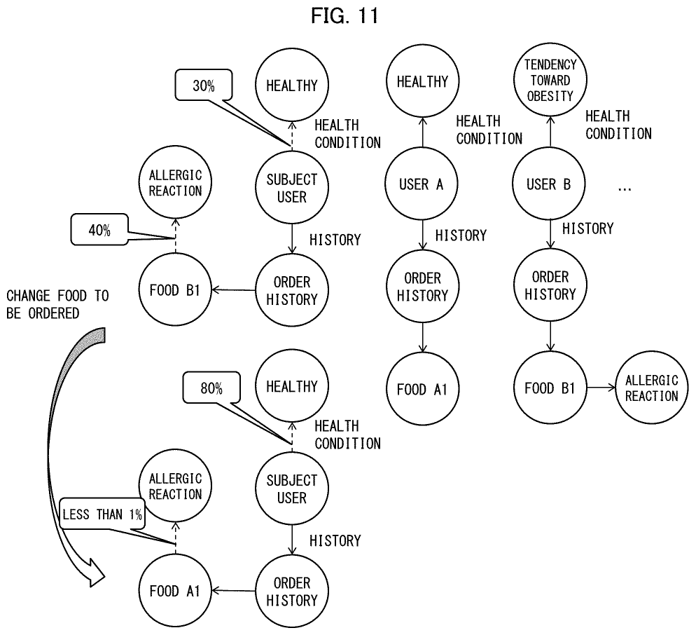

FIG. 11 is a diagram illustrating an overview of a meal recommendation method in accordance with the present example embodiment. In the present example embodiment, an example will be described in which a meal menu conforming to a request is searched while updating a subject user graph including a node indicating an input meal menu.

In the present example embodiment, as in the third example embodiment, link prediction is carried out using a subject user graph and a second user graph. A subject user graph indicated in the upper left part of FIG. 11 includes a node and a link indicating an order history of the subject user and a node and a link indicating a “food B1”.

A graph including a node of a “user A” and a graph including a node of a “user B” illustrated in FIG. 11 are second user graphs. Among those graphs, the second user graph of the user A includes nodes and links indicating that the user A has a health condition “healthy” and that the order history includes a “food A1”. The second user graph of the user B illustrated in FIG. 11 includes nodes and links indicating that the user B has a health condition “tendency toward obesity”, that the order history includes a “food B1”, and that the food B1 causes an allergic reaction. Other nodes and links are not illustrated. At this stage, an order of the “food B1” has not been settled, and the “food B1” is in a state of tentative decision.

By training the second user graphs for various second users as described above, it is possible to carry out link prediction for predicting what meal menu is likely to be related to what health condition or allergic reaction. That is, in the meal menu recommendation method in accordance with the present example embodiment, a tentative subject user graph is generated, and, for example, a probability that a meal menu indicated in the tentative subject user graph is related to a predetermined health condition and a probability that the meal menu causes an allergic reaction are predicted by link prediction.

For example, in the example of FIG. 11, it is predicted, in the upper left subject user graph, that a probability that the node of “healthy” will be connected to the node of “subject user” by the link of “health condition” is 30%. This probability is not high enough. Moreover, it is predicted that a probability that the node of “allergic reaction” will be connected to the node of “food B1” is 40%. This probability is not low enough.

Therefore, as illustrated in the lower left part of FIG. 11, a node connected to the node of “subject user” via the node indicating the order history and a link in the subject user graph is changed from “food B1” to “food A1”, and link prediction is carried out again. Thus, a prediction result of the probability that the node of “healthy” will be connected to the node of “subject user” by the link of “health condition” is changed to 80%. Moreover, it is predicted that a probability that the node of “allergic reaction” will be connected to the node of “food A1” is less than 1%.

According to the meal recommendation method in accordance with the present example embodiment, it is possible to recommend the “food A1” as a meal menu for imparting the health condition of “healthy” to the subject user, based on the result of the above-described process.

### (Apparatus Configuration)

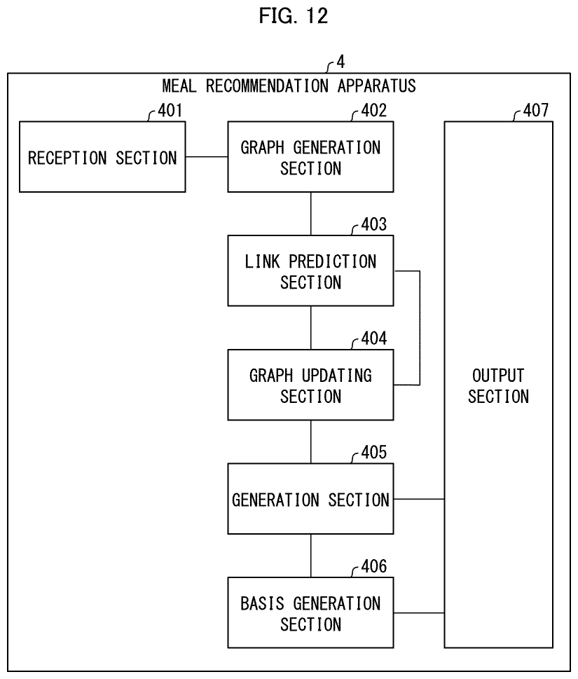

The following description will discuss a configuration of a meal recommendation apparatus 4 in accordance with the fourth example embodiment of the present invention, with reference to FIG. 12. FIG. 12 is a block diagram illustrating the configuration of the meal recommendation apparatus 4 in accordance with the present example embodiment.

As illustrated in FIG. 12, the meal recommendation apparatus 4 includes a reception section 401, a graph generation section 402, a link prediction section 403, a graph updating section 404, a generation section 405, a basis generation section 406, and an output section 407. As with the meal recommendation apparatus 2 in accordance with the second example embodiment, the meal recommendation apparatus 4 may include, in addition to those constituent elements, a learning section, an input apparatus, an output apparatus, a communication apparatus, and the like.

The reception section 401 receives an age, physical information, or a health condition of a subject user and a request pertaining to a meal menu. Here, the request includes information indicating a food menu which has been tentatively decided by the subject user. The request may include information indicating an order history of the subject user, a category of a meal menu (product), a restaurant name, or a store name which the subject user wishes, and the like. The request may include a health condition that the subject user intends, a BMI that the subject user intends, allergy information of the subject user, or the like.

The graph generation section 402 generates a subject user graph based on at least part of the information received by the reception section 401. For example, the graph generation section 402 may generate a subject user graph (see FIG. 11) in which a node indicating the subject user and a node indicating an order history are connected by a link, and the node indicating the order history and a node of “food B1” indicating a food menu which has been tentatively decided by the subject user is connected by a link.

The link prediction section 403 calculates, by link prediction using the learned second user graph and the subject user graph generated by the graph generation section 402, a probability that a predetermined node links to a node included in the subject user graph, the link prediction being carried out for predicting a relationship between nodes which are not connected to each other by a link in the subject user graph and the second user graph. Here, the predetermined node may be identified based on a request or may be determined in advance. For example, in a case where the request indicates that an intended health condition is “healthy”, the link prediction section 403 calculates a probability that a node indicating “healthy” links to a node (e.g., a node of “subject user” in the example of FIG. 11) included in the subject user graph.

The graph updating section 404 updates the subject user graph. For example, the graph updating section 404 carries out a process of replacing a node indicating “food B1” included in the subject user graph with “food A1” which is another meal menu. The graph updating section 404 may carry out a process of adding a node indicating another meal menu.

The updating of the subject user graph may be carried out according to input by a user or may be carried out automatically. In the former case, the graph updating section 404 may cause the output section 407 to output a list of meal menus extracted from the second user graph so that the user selects a new meal menu from among those meal menus. In the latter case, the graph updating section 404 may select a new meal menu candidate from among meal menus extracted from the second user graph.

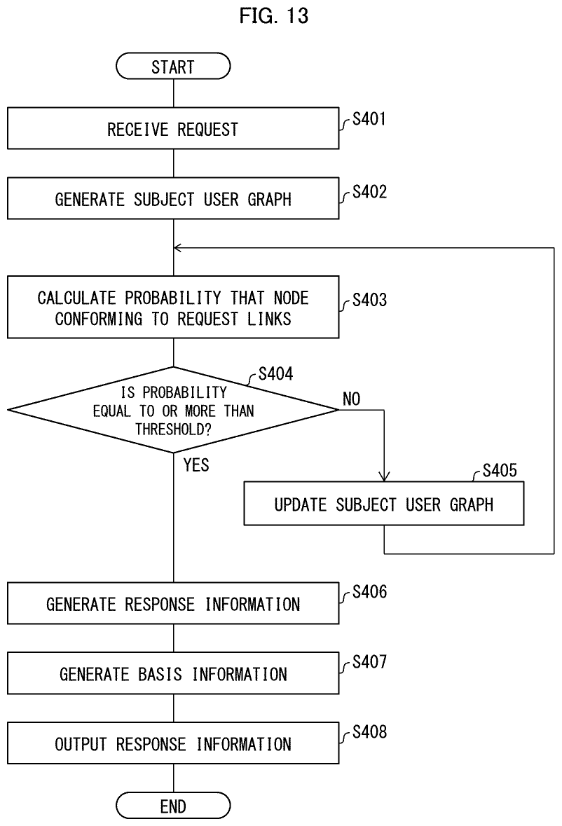

The generation section 405 generates response information including information pertaining to a meal menu which corresponds to the physical information or health condition of the subject user based on the request and a learned model which has learned pieces of physical information or health conditions of a plurality of second users and meal order histories of the plurality of second users. More specifically, the generation section 405 generates response information based on the probability calculated by the link prediction section 403. A specific example of generation of the response information will be described later with reference to FIG. 13.

As described above, the link prediction section 403 carries out link prediction using the second user graphs that are each a learned model and the subject user graph that is generated based on a request. Therefore, the generation section 405 generates response information based on a result of link prediction by the link prediction section 403, and thereby generates response information based on the learned model and the request.

The basis generation section 406 generates basis information indicating validity of response information generated by the generation section 405. Specifically, the basis generation section 406 may generate basis information including at least one selected from the group consisting of a probability that a predetermined health condition links to a node included in the subject user graph and a probability that a node indicating an allergic reaction links to a node included in the subject user graph. The basis generation section 406 may generate basis information pertaining to a result of link prediction by the link prediction section 403 by analyzing the subject user graph and the second user graphs.

The output section 407 outputs, for example, response information generated by the generation section 405. An output destination of the information is not particularly limited, as with the output section 208 in accordance with the second example embodiment.

As described above, the meal recommendation apparatus 4 includes the link prediction section 403 that calculates, by link prediction using the subject user graph and the second user graphs, a probability that a predetermined node links to a node included in the subject user graph. The generation section 405 generates response information based on the probability calculated by the link prediction section 403.

A subject user graph including a node indicating a meal menu input by the subject user can be regarded to indicate a state of the subject user who has purchased the meal menu. Therefore, it can be said that a probability that a predetermined node links to a node included in the subject user graph indicates influence on the subject user by a purchase of the meal menu (i.e., an order of the meal). For example, in a case where a probability that a node indicating that the subject user is healthy links to a node included in the subject user graph including a node indicating that a certain meal menu has been purchased is high, it can be said that a purchase of the certain meal menu has contributed to the health of the subject user.

Therefore, according to the configuration, it is possible to predict, before a meal menu is actually purchased, what kind of influence a purchase of the meal menu is likely to be exerted on the subject user.

### (Flow of Process)

The following description will discuss a flow of a process (meal recommendation method) carried out by the meal recommendation apparatus 4, with reference to FIG. 12. FIG. 12 is a flowchart illustrating the flow of the process carried out by the meal recommendation apparatus 4.

In S401, the reception section 401 receives an age, physical information, or a health condition of a subject user and a request pertaining to a meal menu. In S401, for example, a request is received which includes a meal menu which the subject user wants, a category of the meal menu, a health condition which the subject user intends, allergy information of the subject user, and the like.

In S402, the graph generation section 402 generates a subject user graph based on the information input in S401. Here, the graph generation section 402 generates a subject user graph including at least a node and a link indicating a meal menu which the subject user wants.

In S403, the link prediction section 403 calculates a probability that a node that conforms to the request received in S401 links to a node included in the subject user graph generated in S402. For example, as described above, in a case where the request received in S401 includes a health condition which the subject user intends, a probability that a node indicating the health condition links to a node included in the subject user graph generated in S402 is calculated. As described above, calculation of the probability is carried out by link prediction using the learned second user graphs and the subject user graph. In association with the process of S403, the basis generation section 406 may analyze the subject user graph and the second user graphs to generate basis information indicating a basis of the calculation result by the link prediction section 403.

In S404, the graph updating section 404 determines whether or not the probability calculated in S403 is equal to or more than a threshold. If it has been determined that the probability is equal to or more than the threshold (YES in S404), the process proceeds to S406. If it has been determined that the probability is less than the threshold (NO in S404), the process proceeds to S405.

In a case where properties are included in the request received in S401, for example, prediction is carried out for each of the properties in S403. Then, in S404, the determination may be YES if probabilities for all of the properties are equal to or more than the threshold, and the determination may be NO if any one of the characters is less than the threshold. In a case where allergy information is included in the request received in S401, the process may proceed to S406 if a probability that a node indicating an allergic reaction corresponding to the allergy information links to a node included in the subject user graph is less than a predetermined threshold, and the process may proceed to S405 if the probability is equal to or more than the threshold.

In S405, the graph updating section 404 updates the subject user graph. Specifically, the graph updating section 404 replaces a node of a meal menu included in the current subject user graph with a node of another meal menu. The update content may be decided according to input by a user, or may be decided by the graph updating section 404, as described above.

When the subject user graph has been updated, the process returns to S403, and calculation of a probability is carried out again. That is, in the process of FIG. 15, until it is determined to be YES in S404, calculation of a probability in S403 and updating of the subject user graph in S405 are repeatedly carried out.

In S406, the generation section 405 infers a meal menu that conforms to the request received in S401, and generates response information indicating the inferred meal menu. Specifically, the generation section 405 infers that a meal menu at the time when it is determined to be YES in S404 is a meal menu that conforms to the request, and generates response information indicating that meal menu.

In S407, the basis generation section 406 generates basis information indicating validity of the response information generated in S406. Specifically, the basis generation section 406 generates basis information including at least one selected from the group consisting of a probability that a predetermined health condition links to a node included in the subject user graph and a probability that a node indicating an allergic reaction links to a node included in the subject user graph.

In S408, the output section 407 outputs the response information which has been generated in S406. At this time, the output section 407 may output also the basis information which has been generated in S407. Thus, the process of FIG. 13 ends.

### [Variation]

As described in the fourth example embodiment, by using the subject user graph and the second user graph, it is possible to predict, by link prediction, a probability that a node of a subject user including a certain meal menu as a node links to a node indicating a predetermined property. Therefore, for example, it is possible to predict, before a meal menu is actually purchased, what kind of influence a purchase of the meal menu is likely to be exerted on the subject user.

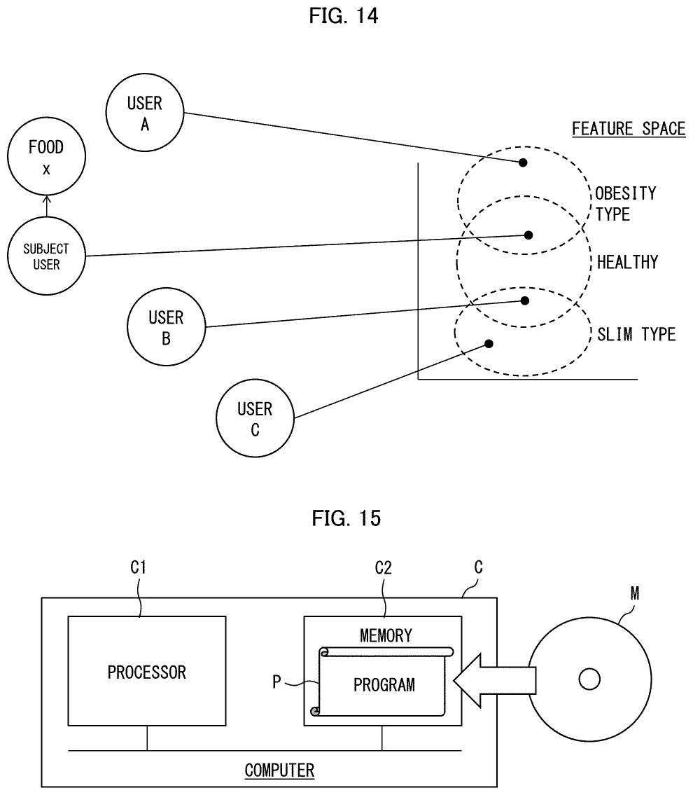

Here, prediction of a relevance between the subject user and a predetermined property can also be carried out by a method other than link prediction. This will be described with reference to FIG. 14. FIG. 14 is a diagram illustrating an example of predicting a property of a subject user based on a feature quantity calculated from a subject user graph and second user graphs. FIG. 14 shows second user graphs of users A through C and a subject user graph of a subject user. Note that, among nodes and links included in these graphs, those other than nodes and links indicating that a food menu which the subject user is considering to order is a food x are not illustrated.

Here, it is possible to calculate a feature quantity for each second user by multiplying a feature quantity of each node included in the second user graph by a weight corresponding to a link connected to that node, and adding up the weighted feature quantities. Therefore, by carrying out learning in which the weight is updated so that a calculated feature quantity corresponds to a character of the second user, it is possible to predict a property of a subject user from a feature quantity of a subject user graph which has been calculated while applying the weight.

For example, in the example of FIG. 14, a feature quantity calculated from the second user graph of the user A who has been found to be of an obesity type is learned to fall within a range corresponding to a property “obesity type” in a feature space. Moreover, a feature quantity calculated from the second user graph of the user B who has been found to be healthy is learned to fall within a range corresponding to a property “healthy” in the feature space. Similarly, a feature quantity calculated from the second user graph of the user C who has been found to be of a slim type is learned to fall within a range corresponding to a property “slim type” in the feature space.

In this case, if a feature quantity calculated from the subject user graph is included within the range corresponding to the properties “obesity type” and “healthy” as illustrated in FIG. 14, it is possible to predict that the subject user has the property “obesity type” or “healthy”. Such a property prediction method can be applied as an alternative method to the method of property prediction in the above-described example embodiments.

That is, in the example of FIG. 14, it is predicted that, if the subject user orders the food x, the subject user will have a health condition of “obesity type” to “healthy”. If a food to be linked to the subject user graph is changed, the prediction result can also change. Therefore, it is possible to detect, while changing a food to be linked to the subject user graph, a food for which a prediction result is an intended property (e.g., “healthy”) as in the fourth example embodiment.

### [Software Implementation Example]

Some or all of the functions of each of the meal recommendation apparatuses 1 through 4 (hereinafter, referred to as “present apparatus”) may be implemented by hardware such as an integrated circuit (IC chip), or may be implemented by software.

In the latter case, the present apparatus is realized by, for example, a computer that executes instructions of a program that is software realizing the foregoing functions. FIG. 15 illustrates an example of such a computer (hereinafter, referred to as “computer C”). The computer C includes at least one processor C1 and at least one memory C2. The memory C2 stores a program (meal recommendation program) P for causing the computer C to function as the present apparatus. In the computer C, the processor C1 reads the program P from the memory C2 and executes the program P, so that the functions of the present apparatus are realized.

Examples of the processor C1 include a central processing unit (CPU), a graphic processing unit (GPU), a digital signal processor (DSP), a micro processing unit (MPU), a floating point number processing unit (FPU), a physics processing unit (PPU), a microcontroller, and a combination thereof. Examples of the memory C2 include a flash memory, a hard disk drive (HDD), a solid state drive (SSD), and a combination thereof.

Note that the computer C can further include a random access memory (RAM) in which the program P is loaded when the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display and a printer.

The program P can be stored in a computer C-readable, non-transitory, and tangible storage medium M. The storage medium M can be, for example, a tape, a disk, a card, a semiconductor memory, a programmable logic circuit, or the like. The computer C can obtain the program P via the storage medium M. The program P can be transmitted via a transmission medium. The transmission medium can be, for example, a communication network, a broadcast wave, or the like. The computer C can obtain the program P also via such a transmission medium.

### [Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### [Additional Remark 2]

Some or all of the foregoing example embodiments can also be described as below. Note, however, that the present invention is not limited to the following supplementary notes.

### (Supplementary Note 1)

A meal recommendation apparatus including: a reception means for receiving physical information or a health condition of a subject user and a request pertaining to a meal menu; a generation means for generating response information including information pertaining to a meal menu which corresponds to the physical information or health condition of the subject user based on the request and a learned model which has learned pieces of physical information or health conditions of a plurality of second users and meal order histories of the plurality of second users; and an output means for outputting the response information. According to the configuration, it is possible to bring about an example advantage of recommending a meal menu that is suitable for the subject user, even if the subject user himself/herself does not recognize suitability.

### (Supplementary Note 2)

The meal recommendation apparatus according to supplementary note 1, further including: a basis information generation means for generating basis information including information pertaining to a person whose physical information or health condition is similar to that of the subject user among the plurality of second users, the output means further outputting the basis information. According to the configuration, the user can refer to response information while taking into consideration a basis thereof.

### (Supplementary Note 3)

The meal recommendation apparatus according to supplementary note 1 or 2, in which: the learned model is a second user graph that is a graph which includes (i) a node indicating a second user, who is different from the subject user, (ii) nodes each indicating physical information, a health condition, or a meal menu related to the second user, and (iii) links indicating relationships between the nodes, and which has learned the relationships between the nodes. According to the configuration, it is possible to recommend a proper meal menu while taking into consideration a mutual relationship between physical information, a health condition, and a meal menu related to a second user.

### (Supplementary Note 4)

The meal recommendation apparatus according to supplementary note 3, further including: a link prediction means for predicting, by link prediction using a subject user graph and the second user graph, a node which links to a node included in the subject user graph from among nodes which are included in the second user graph and indicate meal menus related to the second user, the subject user graph including a plurality of nodes pertaining to the subject user, and the link prediction being carried out for predicting a relationship between nodes which are not connected to each other by a link in the subject user graph and the second user graph, the generation means generating response information including information pertaining to a meal menu which corresponds to the node predicted by the link prediction means. According to the configuration, it is possible to recommend, to the subject user, a meal menu that is suitable for the subject user, while taking into consideration useful information.

### (Supplementary Note 5)

The meal recommendation apparatus according to supplementary note 4, in which: the reception means receives input of a condition for the second user graph; and the link prediction means predicts, from among nodes that indicate meal menus and are included in a second user graph satisfying the condition, a node which links to a node included in the subject user graph. According to the configuration, the prediction range of a node that links to the subject user graph is narrowed down to a second user graph that satisfies the input condition. Therefore, it is possible to predict a node conforming to an intention of the subject user.

### (Supplementary Note 6)

The meal recommendation apparatus according to supplementary note 4, further including: an evaluation means for evaluating, based on another node included in the second user graph including the node predicted by the link prediction means, a recommendation level, for the subject user, of a meal menu indicated by the node. According to the configuration, it is possible to contribute to purchase of a meal menu which is more favorable for the subject user.

### (Supplementary Note 7)

The meal recommendation apparatus according to supplementary note 3, further including: a link prediction means for identifying a second user who has a predetermined relationship with the subject user by link prediction using a subject user graph and a plurality of second user graphs, the subject user graph including a plurality of nodes pertaining to the subject user, the plurality of second user graphs having been respectively generated for a plurality of second users, and the link prediction being carried out for predicting a relationship between nodes which are not connected to each other by a link in the subject user graph and the plurality of second user graphs, the generation means generating response information including information pertaining to a meal menu related to the second user who has been identified by the link prediction means. According to the configuration, it is possible to carry out recommendation suitable for the subject user.

### (Supplementary Note 8)

The meal recommendation apparatus according to supplementary note 7, in which: the link prediction means identifies a second user who is similar to the subject user; and the generation means generates response information that recommends, to the subject user, a meal menu indicated by a node included in a second user graph of the second user who has been identified by the link prediction means. According to the configuration, it is possible to recommend, to the user, a meal menu that is highly likely to be suitable for the subject user.

### (Supplementary Note 9)

The meal recommendation apparatus according to supplementary note 3, further including: a reception means for receiving input of a meal menu by the subject user; and a link prediction means for calculating, by link prediction using the second user graph and a subject user graph including a node indicating the meal menu which has been input, a probability that a predetermined node links to a node included in the subject user graph, the link prediction being carried out for predicting a relationship between nodes which are not connected to each other by a link in the subject user graph and the second user graph, the generation means generating response information based on the probability which has been calculated by the link prediction means. According to the configuration, it is possible to predict, before a meal menu is actually purchased, what kind of influence a purchase of the meal menu is likely to be exerted on the subject user.

### (Supplementary Note 10)

A meal recommendation method including: receiving, by a computer, physical information or a health condition of a subject user and a request pertaining to a meal menu; generating, by the computer, response information including information pertaining to a meal menu which corresponds to the physical information or health condition of the subject user based on the request and a learned model which has learned pieces of physical information or health conditions of a plurality of second users and meal order histories of the plurality of second users; and outputting, by the computer, the response information. According to the method, it is possible to bring about an example advantage of recommending a meal menu that is suitable for the subject user, even if the subject user himself/herself does not recognize suitability.

### (Supplementary Note 11)

A meal recommendation program for causing a computer to carry out: a process of receiving physical information or a health condition of a subject user and a request pertaining to a meal menu; a process of generating response information including information pertaining to a meal menu which corresponds to the physical information or health condition of the subject user based on the request and a learned model which has learned pieces of physical information or health conditions of a plurality of second users and meal order histories of the plurality of second users; and a process of outputting the response information. According to the program, it is possible to bring about an example advantage of recommending a meal menu that is suitable for the subject user, even if the subject user himself/herself does not recognize suitability.

### [Additional Remark 3]

Furthermore, some of or all of the foregoing example embodiments can also be expressed as below.

A meal recommendation apparatus including at least one processor, the at least one processor carrying out: a process of receiving physical information or a health condition of a subject user and a request pertaining to a meal menu; a process of generating response information including information pertaining to a meal menu which corresponds to the physical information or health condition of the subject user based on the request and a learned model which has learned pieces of physical information or health conditions of a plurality of second users and meal order histories of the plurality of second users; and a process of outputting the response information.

The meal recommendation apparatus may further include a memory. The memory may store a program (meal recommendation program) for causing the at least one processor to carry out: a process of receiving physical information or a health condition of a subject user and a request pertaining to a meal menu; a process of generating response information including information pertaining to a meal menu which corresponds to the physical information or health condition of the subject user based on the request and a learned model which has learned pieces of physical information or health conditions of a plurality of second users and meal order histories of the plurality of second users; and a process of outputting the response information. The program can be stored in a computer-readable non-transitory tangible storage medium.

### REFERENCE SIGNS LIST

1: Meal recommendation apparatus
11: Reception section
12: Generation section
13: Output section
2: Meal recommendation apparatus
201: Reception section
202: Graph generation section
203: Learning section
204: Link prediction section
205: Evaluation section
206: Generation section
207: Basis generation section
208: Output section
3: Meal recommendation apparatus
301: Reception section
302: Graph generation section
303: Link prediction section
304: Evaluation section
305: Generation section
306: Basis generation section
4: Meal recommendation apparatus
401: Reception section
402: Graph generation section
403: Link prediction section
404: Graph updating section
405: Evaluation section
406: Basis generation section

What is claimed is:

1. A meal recommendation apparatus comprising at least one processor, the at least one processor carrying out:
a reception process of receiving physical information or a health condition of a subject user and a request pertaining to a meal menu;
a generation process of generating response information including information pertaining to a meal menu which corresponds to the physical information or health condition of the subject user based on the request and a learned model which has learned pieces of physical information or health conditions of a plurality of second users and meal order histories of the plurality of second users; and
an output process of outputting the response information.

2. The meal recommendation apparatus according to claim 1, wherein:
the at least one processor further carries out a basis information generation process of generating basis information including information pertaining to a person whose physical information or health condition is similar to that of the subject user among the plurality of second users,
in the output process, the at least one processor further outputs the basis information.

3. The meal recommendation apparatus according to claim 1, wherein:
the learned model is a second user graph that is a graph which includes (i) a node indicating a second user, who is different from the subject user, (ii) nodes each indicating physical information, a health condition, or a meal menu related to the second user, and (iii) links indicating relationships between the nodes, and which has learned the relationships between the nodes.

4. The meal recommendation apparatus according to claim 3, wherein:
the at least one processor further carries out a link prediction process of predicting, by link prediction using a subject user graph and the second user graph, a node which links to a node included in the subject user graph from among nodes which are included in the second user graph and indicate meal menus related to the second user, the subject user graph including a plurality of nodes pertaining to the subject user, and the link prediction being carried out for predicting a relationship between nodes which are not connected to each other by a link in the subject user graph and the second user graph; and
in the generation process, the at least one processor generates response information including information pertaining to a meal menu which corresponds to the node predicted in the link prediction process.

5. The meal recommendation apparatus according to claim 4, wherein:
in the reception process, the at least one processor receives input of a condition for the second user graph; and
in the link prediction process, the at least one processor predicts, from among nodes that indicate meal menus and are included in a second user graph satisfying the condition, a node which links to a node included in the subject user graph.

6. The meal recommendation apparatus according to claim 4, wherein:
the at least one processor further carries out an evaluation process of evaluating, based on another node included in the second user graph including the node predicted by in the link prediction process, a recommendation level, for the subject user, of a meal menu indicated by the node.

7. The meal recommendation apparatus according to claim 3, wherein:
the at least one processor further carries out a link prediction process of identifying a second user who has a predetermined relationship with the subject user by link prediction using a subject user graph and a plurality of second user graphs, the subject user graph including a plurality of nodes pertaining to the subject user, the plurality of second user graphs having been respectively generated for a plurality of second users, and the link prediction being carried out for predicting a relationship between nodes which are not connected to each other by a link in the subject user graph and the plurality of second user graphs; and
in the generation process, the at least one processor generates response information including information pertaining to a meal menu related to the second user who has been identified in the link prediction process.

8. The meal recommendation apparatus according to claim 7, wherein:
in the link prediction process, the at least one processor identifies a second user who is similar to the subject user; and
in the generation process, the at least one processor generates response information that recommends, to the subject user, a meal menu indicated by a node included in a second user graph of the second user who has been identified in the link prediction process.

9. The meal recommendation apparatus according to claim 3, wherein:
the at least one processor further carries out a reception process of receiving input of a meal menu by the subject user;
the at least one processor further carries out a link prediction process of calculating, by link prediction using the second user graph and a subject user graph including a node indicating the meal menu which has been input, a probability that a predetermined node links to a node included in the subject user graph, the link prediction being carried out for predicting a relationship between nodes which are not connected to each other by a link in the subject user graph and the second user graph; and
in the generation process, the at least one processor generates response information based on the probability which has been calculated in the link prediction process.

10. A meal recommendation method comprising:
receiving, by a computer, physical information or a health condition of a subject user and a request pertaining to a meal menu;
generating, by the computer, response information including information pertaining to a meal menu which corresponds to the physical information or health condition of the subject user based on the request and a learned model which has learned pieces of physical information or health conditions of a plurality of second users and meal order histories of the plurality of second users; and
outputting, by the computer, the response information.

11. A computer-readable non-transitory storage medium storing a meal recommendation program for causing a computer to carry out:
a process of receiving physical information or a health condition of a subject user and a request pertaining to a meal menu;
a process of generating response information including information pertaining to a meal menu which corresponds to the physical information or health condition of the subject user based on the request and a learned model which has learned pieces of physical information or health conditions of a plurality of second users and meal order histories of the plurality of second users; and
a process of outputting the response information.
